(19) United States
(12) Patent Application Publication
(10) Pub. No.: US 2010/0210987 A1
(43) Pub. Date: Aug. 19, 2010

(54) BREATHABLE MEDICAL ADHESIVE TAPE AND MANUFACTURING METHOD THEREOF

(76) Inventor: HSIN-KAI LU, Xinzhuang City (TW)

Correspondence Address: LEONG C LEI, PMB # 1008, 1867 YGNACIO VALLEY ROAD, WALNUT CREEK, CA, 94598, US

(21) Appl. No.: 12/371,922

(22) Filed: Feb. 17, 2009

(51) Int. Cl. A61F 13/02

(52) U.S. Cl. 602/54; 427/2.31

(57) ABSTRACT

A breathable medical adhesive tape includes an air-permeable substrate, such as a non-woven fabric, a cotton fabric, or a nylon sheet having air pores, having a surface on which a layer of adhesive is applied. A plurality of continuous non-adhesive-coated air-transmittable patterns is formed in the adhesive layer of the air-permeable substrate. The non-adhesive-coated air-transmittable patterns are formed by employing a scrape mechanism to scrape off a portion of the adhesive from the adhesive layer of the air-permeable substrate. The adhesive tape is advantageous in more effective dry-keeping, enhanced adhesion, and reduced allergy caused by excessive adhesive.

### TECHNICAL FIELD OF THE INVENTION

The present invention generally relates to a technique related to the formation and structure of an air-breathable adhesive tape that is applicable in surgical therapy for covering a patient's skin, which can be widely used in medical purpose adhesive tape, such as a roll of breathable adhesive tape, a wound bandage (Band-Aid) or a wound pad.

### DESCRIPTION OF THE PRIOR ART

Adhesive tapes, such as medical adhesive tapes, including breathable adhesive tape rolls, plastic bandages (Band-Aid), or wound pads, are commonly used in surgical therapy for medical purposes to adhesively attach to a patient's skin for fixing a wound or covering acnes of small sizes. The adhesive tape is comprised of a substrate having a surface to which adhesive is applied for covering and attaching to the skin for fixing purposes. Although the known adhesive tape is effective in attaching to the skin to provide fixing and protection, since the adhesive tape is fixed to the skin through the adhesives, the portion of the skin covered by the tape is often susceptible to getting inflamed, allergy, and itching. This is common experience to most people who have used the known medical adhesive tapes.

To enhance air breathing of the medical adhesive tape, a substrate of the adhesive tape is often made of an air-permeable material, such as non-woven fabric, cotton fabric, or a nylon sheet with air pores. However, since a surface of the substrate is completely coated with adhesive, air breathing of the substrate material is substantially affected by the adhesive. Apparently, the conventional adhesive tape does not possess excellent air breathing. Thus, when an adhesive tape that is applied to a patient's skin is wetted by for example humidity and sweat, the humidity or sweat will remain on the skin and cannot be effectively dissipated. This lowers the adhesion of the tape, leading to undesired peeling off and separation of the tape. Even worse, the skin may get seriously pale and corrugated due to being long term soaked in water/sweat, and this leads to propagation of bacteria and inflaming of wounds, eventually making the wound not closing up. These are just some of the disadvantages of the convention adhesive tapes.

Full coverage type of known bandages, adhesive tapes, and gauzes, such as that disclosed in Japanese Patent No. 3-75055, use woven fabric or non-woven fabric with an inner side having a plastic film for covering a wound, to allow the known bandage or adhesive tape to attach to and comply with the skin. Such a design, although providing excellent compliance of surgical adhesive tapes, has poor air permeability and is thus functionally non-satisfactory.

Thus, it is desired to provide an adhesive tape that offers excellent air breathing while having excellent adhesion to skin to comply with the need for skin caring for patients.

### SUMMARY OF THE INVENTION

The primary objective of the present invention is to provide a breathable medical adhesive tape comprising an air-permeable substrate, such as a non-woven fabric, a cotton fabric, or a nylon sheet having air pores, having a surface on which a layer of adhesive is applied. A plurality of continuous non-adhesive-coated air-transmittable patterns is formed in the adhesive layer of the air-permeable substrate. The non-adhesive-coated air-transmittable patterns are formed by employing a scrape mechanism to scrape off a portion of the adhesive from the adhesive layer of the air-permeable substrate to provide the breathable adhesive tape with a plurality of non-adhesive-coated air-transmittable patterns that enhance air breathing of the adhesive tape. The adhesive tape is advantageous in more effective dry-keeping, enhanced adhesion, and reduced allergy caused by adhesives.

The foregoing objective and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a perspective view of the present invention.

FIG. 2 schematically illustrates the flow of a manufacturing method in accordance with the present invention.

FIG. 3 schematically illustrates the major step of manufacturing in accordance with the present invention.

FIG. 4 schematically illustrates the flow of an alternative manufacturing method in accordance with the present invention.

FIG. 5 illustrates an application of the present invention in a wound pad.

FIG. 6 illustrates an application of the present invention in a plastic bandage.

FIG. 7 demonstrates the advantages of the present invention applied to a user's skin.

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

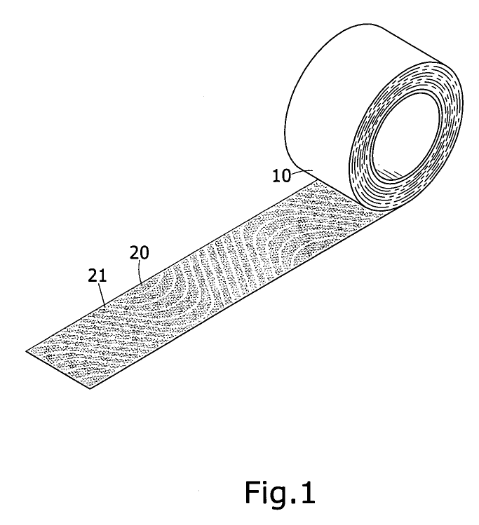

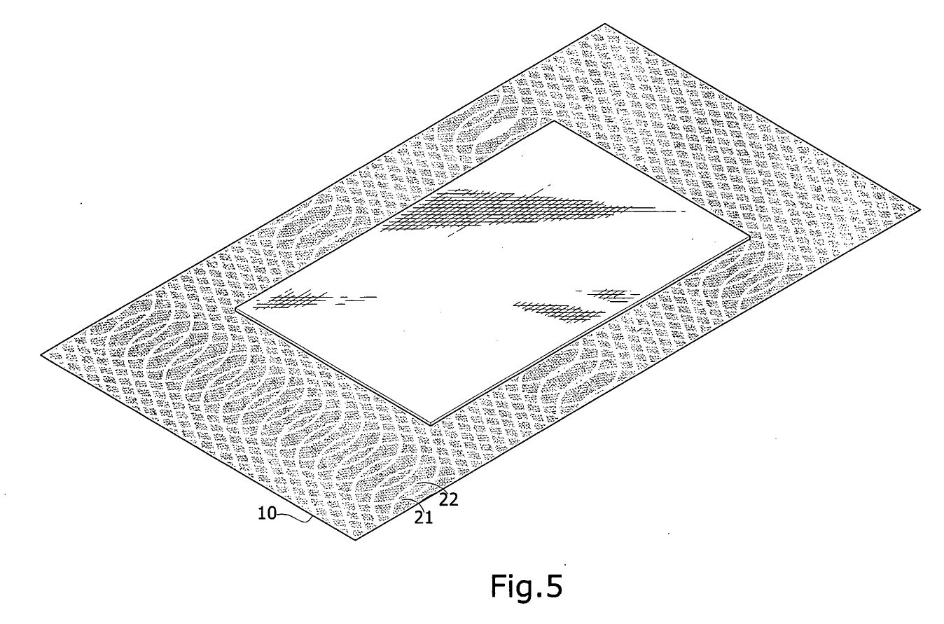

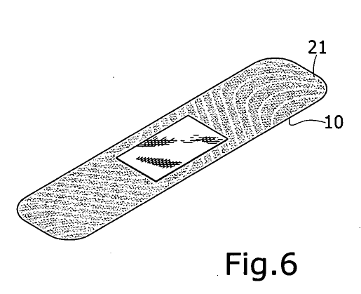

With reference to the drawings and in particular to FIG. 1, a breathable medical adhesive tape constructed in accordance with the present invention comprises a base that is constituted by an air permeable substrate 10, such as non-woven fabric, cotton fabric, and a nylon sheet with air pores and a layer of adhesive 20 coated on a surface of the substrate 10. The adhesive layer 20 of the air permeable substrate 10 forms a plurality of continuous non-adhesive-coated air-transmittable patterns 21. Preferably, the non-adhesive-coated air-transmittable patterns 21 are formed of continuous wavy patterns, which are regarded as a basic and preferred manner for practicing the present invention, and which is advantageous in providing excellent adhesion for cutting applied in any direction on the tape, so as to ensure no undesired peeling and separation of the tape. Referring to FIG. 5, an embodiment where two continuous non-adhesive-coated air-transmittable patterns 21, 22 that intersect each other are formed. It is apparent that the continuous wavy patterns can be replaced by interconnected line segments, including two sets of interconnected line segments that intersect each other to define a rhombus grating configuration (not shown), which is regarded as an equivalent way of practicing the present invention. The breathable adhesive tape of the present invention can be widely applied to for example a breathable adhesive tape roll as shown in FIG. 1, or a wound pad as shown in FIG. 5, or a plastic bandage (band-aid) as shown in FIG. 6, or similar medical adhesive tapes.

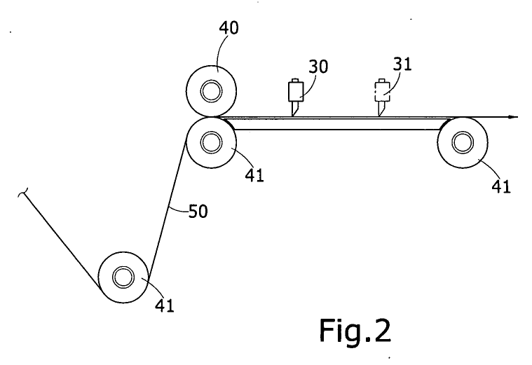

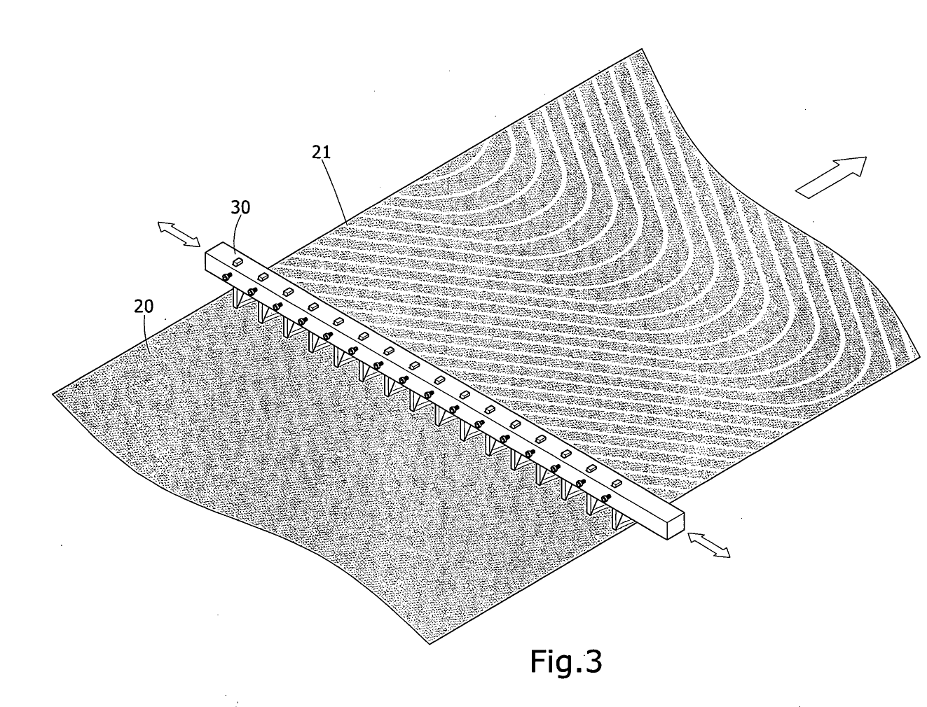

The method for manufacturing the breathable adhesive tape of the present invention will be described with reference to FIGS. 2 and 3.

(1) Providing a substrate material of a suitable width and applying adhesive thereto: Guide rollers 41 are properly set in front of a roll of substrate material 50 and an adhesive application roller 40 is arranged to uniformly apply a layer of adhesive 20 on a surface of the substrate 50.

(2) Forming non-adhesive-coated air-transmittable patterns 21 on the substrate 20 that is coated with the adhesive 20: A scrape mechanism 30 is provided behind the adhesive application roller 40. The scrape mechanism 30 carries a palisade structure of multiple blades that is reciprocally movable in a direction substantially normal to a forwarding direction of the substrate 50 to scrape off a portion of the adhesive 20 for forming the wavy configuration of continuous non-adhesive-coated air-transmittable patterns 21.

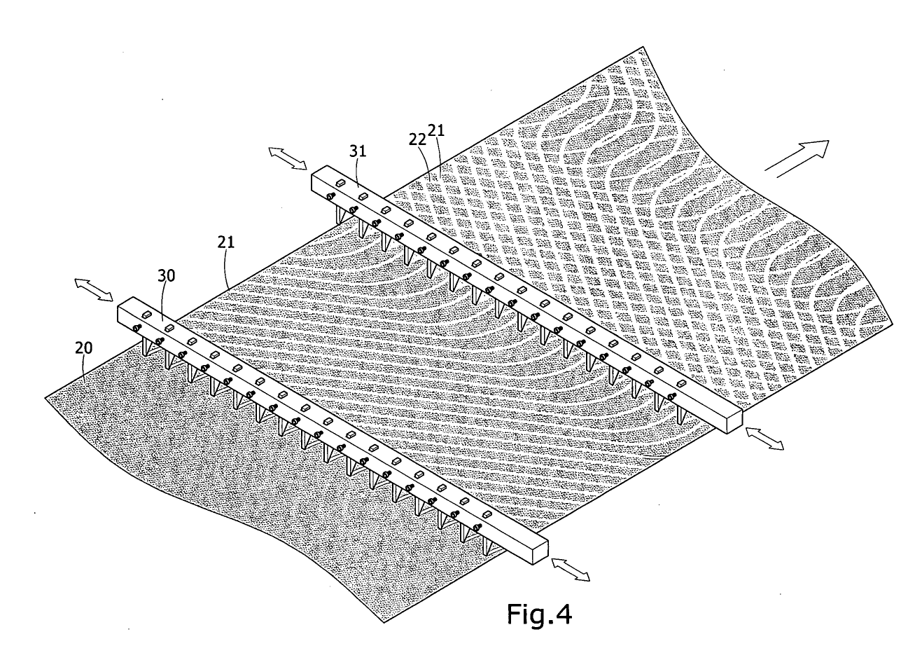

In an alternative way, as shown in FIGS. 2 and 4, two or more sets of scrape mechanisms 30, 31 are set to space from each other and are both reciprocally movable in a direction substantially normal to a forwarding direction of the substrate 50 to scrape off a portion of the adhesive 20 for forming two sets wavy configuration of continuous non-adhesive-coated air-transmittable patterns 21, 22, as shown in FIG. 5.

Since the directions of moving of the scrape mechanisms 30, 31 and the forwarding direction of the substrate 50 are changeable, it is possible to form, if desired, interconnected line segments on the adhesive layer 20, including two sets of interconnected line segments that intersect each other to define a rhombus grating configuration (not shown), which is regarded as an equivalent way of practicing the present invention. The breathable adhesive tape of the present invention can be widely applied to for example a breathable adhesive tape roll as shown in FIG. 1, or a wound pad as shown in FIG. 5, or a plastic bandage (band-aid) as shown in FIG. 6, or similar medical adhesive tapes.

(3) Cutting: After being subjected to cutting, the tape of the present invention can be used in various applications and packages of breathable adhesive tapes.

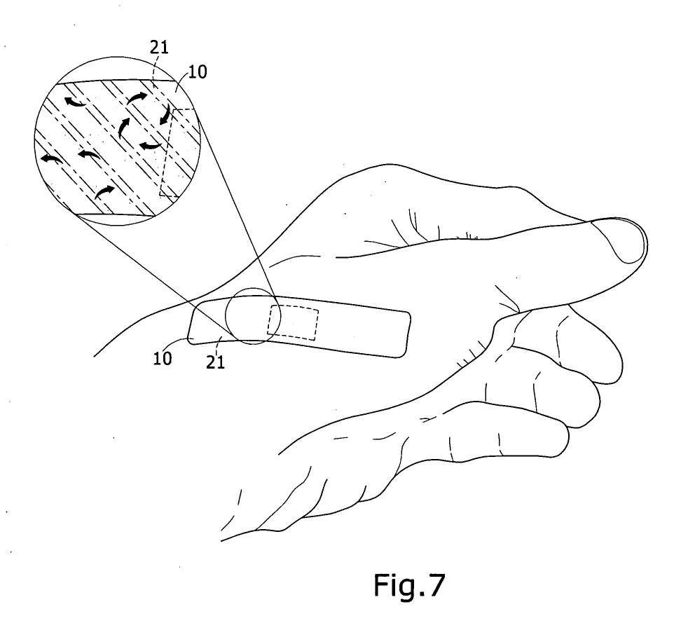

Referring to FIG. 7, in the use of the breathable adhesive tape of the present invention, since a plurality of continuous non-adhesive-coated patterns (21) are provided, a user's skin is provided with sufficient space for air transmission, whereby sweat, water, and heat can be expelled out through the continuous non-adhesive-coated patterns 21, making the skin dry and allowing a wound or acne to easily close up. Since humidity is efficiently dissipated, the adhesion is made stronger and firmer. Further, since the quantity of adhesive used is lessened and thus more dried, the user's skin is not susceptible to allergy. These are the advantages and benefits of the present invention.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. A breathable medical adhesive tape, comprising an air permeable substrate having a surface on which a layer of adhesive is coated, the adhesive layer forming a plurality of continuous non-adhesive-coated air-transmittable patterns, which are of a wavy and continuous configuration.

2. The breathable medical adhesive tape according to claim 1, wherein the air permeable substrate comprises non-woven fabric.

3. The breathable medical adhesive tape according to claim 1, wherein the air permeable substrate comprises cotton fabric.

4. The breathable medical adhesive tape according to claim 1, wherein the air permeable substrate comprises a nylon sheet with air pores

5. The breathable medical adhesive tape according to claim 1, wherein the non-adhesive-coated air-transmittable patterns comprise wavy patterns intersecting each other.

6. A breathable medical adhesive tape, comprising an air permeable substrate having a surface on which a layer of adhesive is coated, the adhesive layer forming a plurality of continuous non-adhesive-coated air-transmittable patterns, which are of a rhombus grating configuration.

7. A method for manufacturing a breathable medical adhesive tape, comprising the following steps:
(1) providing a substrate material of a suitable width and applying adhesive thereto, wherein a guide roller is set in front of a roll of substrate material and an adhesive application roller is arranged to uniformly apply a layer of adhesive on a surface of the substrate;
(2) forming non-adhesive-coated air-transmittable patterns on the substrate that is coated with the adhesive, wherein a scrape mechanism is provided behind the adhesive application roller, the scrape mechanism being reciprocally movable in a direction that is not coincident to a forwarding direction of the substrate to scrape off a portion of the adhesive for forming a wavy configuration of continuous non-adhesive-coated air-transmittable patterns; and
(3) cutting: after being subjected to cutting, various applications of breathable adhesive tape are selectively carried out.

8. The method according to claim 7, wherein two scrape mechanisms are respectively arranged at front and rear sides.
